(19) United States
(12) Patent Application Publication
(10) Pub. No.: US 2011/0111040 A1
(43) Pub. Date: May 12, 2011

(54) POLYELECTROLYTE-ENCAPSULATED GOLD NANOPARTICLES CAPABLE OF CROSSING BLOOD-BRAIN BARRIER

(76) Inventors: Silke Krol, Basovizza(Trieste) (IT); Julian Lopez-Viota Gallardo, Basovizza(Trieste) (IT)

(21) Appl. No.: 12/993,924

(22) PCT Filed: May 19, 2009
(86) PCT No.: PCT/EP2009/056042

§ 371 Date: Dec. 27, 2010

(30) Foreign Application Priority Data
May 20, 2008 (EP) 08425351.7

(51) Int. Cl. A61K 9/50; A61K 31/205; A61P 25/28; A61P 25/16; B82Y 5/00

(52) U.S. Cl. 424/491; 514/556; 977/773; 977/906

(57) ABSTRACT

A gold-creatine nanoparticle is described, preferably covered with albumin, together with a process for its preparation and its use as medicament, in particular for the treatment of stroke. Said gold nanoparticle is capable of crossing the blood-brain barrier.

### TECHNICAL FIELD

The present invention refers to the medical field, in particular to pharmaceutical formulations for the selective release of drugs.

More in particular, the present invention refers to nanoparticles, to a process for their preparation and to their use as a medicament to treat diseases and conditions that require that the pharmaceutical agent, in particular creatine, is delivered through one or more physiological barriers, in particular the blood-brain barrier.

### STATE OF THE ART

One of the main tasks in the development of advanced pharmaceutical formulations is the production of delivery systems capable of providing a targeted release of drugs.

There is a need to ensure that these transport systems reach certain sites and should therefore be able to cross the physiological barriers, including the blood-brain barrier.

The nanoparticles are used for the controlled release and the delivery of active pharmaceutical ingredients to specific sites and possess a great potential in many areas of medicine.

WO 2007/012114 A discloses the use of gold nanoparticles as delivery means for a large spectrum of therapeutic agents which includes peptides and proteins.

WO 2008/070171 A discloses the use of nanoparticles, in particular gold nanoparticles optionally derivatised with either a polymer coating, cyclodextran or a SAM for the delivery of pharmaceutical agents such as proteins and peptides to the desired site of action.

WO 2006/102377 A discloses nanoparticles made of metal cores and encapsulated in an albumin matrix, which are able to cross the blood-brain barrier.

EP-A-1 815 851 discloses nanoparticles formed by albumin to be used as delivery carrier of pharmaceutical agents through the blood-brain barrier.

US 2006/073210 A1 discloses that nanoparticles which surface is dominated by chitosan are delivery means of pharmaceutical agent with improved blood-brain barrier penetration.

There is also the need that said release systems are stable in physiological conditions.

Creatine is a guanidine compound endogenously produced by liver, kidney and pancreas (Juhn M. S., Tarnopolsky M., Oral creatine supplementation and athletic performance: a critical review. Clin J Sport Med 1998; 8:286-297).

Creatine is known to increase muscle and brain phosphocreatine concentrations, and may inhibit the activation of the mitochondrial permeability transition, protects against neuronal degeneration in transgenic murine models of amyotrophic lateral sclerosis and Huntington's disease and in chemically mediated neurotoxicity (Tarnopolsky, M. A., et al., Potential for creatine and other therapies targeting cellular energy dysfunction in neurological disorders, Ann Neurol 2001; 49:561-574).

US 2001/006989 A1 discloses nanoparticles which contain creatine phosphate as biologically active molecule for therapeutic use.

The need to get creatine in the cerebral stroke site still exists because the blood-brain barrier is one of the physiological barriers more difficult to cross.

There are pharmaceutical compositions and methods to increase the delivery of drugs and other agents through the blood-brain barrier (U.S. Pat. No. 6,419,949, WO 89/11299, US 2002/115747, WO 02/69930, WO 2006/44660 and WO 2007/88066).

All these methods adopt different systems and no one refers to gold-creatine nanoparticle.

An object of the invention is a system for transporting creatine through the blood-brain barrier that is capable of carrying a therapeutically effective dose and is stable under physiological conditions.

### SUMMARY OF THE INVENTION

It has now been found that gold nanoparticles are capable of delivering creatine through the blood-brain barrier, in particular when covered by an albumin layer.

Gold-creatine nanoparticles are an object of the present invention.

It is another object of the present invention a process for their preparation.

Further objects of the present invention are nanoparticles for use as a medicament, in particular for the treatment of stroke.

It is another object of the present invention a pharmaceutical composition comprising an effective amount of said nanoparticles.

These and other objects of the invention will be described in detail also by examples and figures.

In the figures:

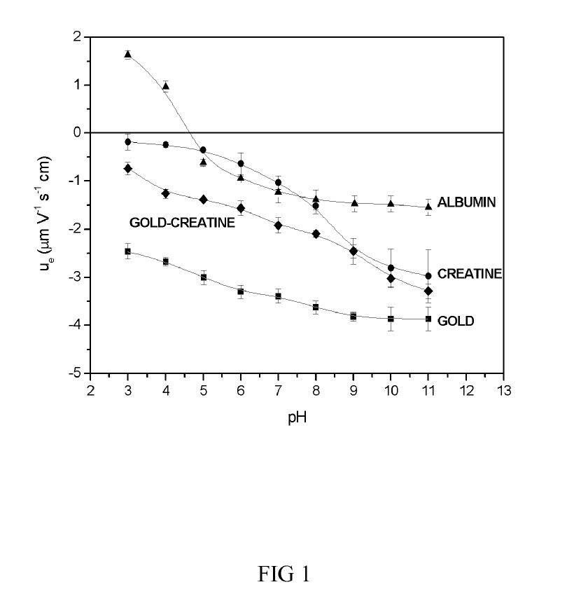

FIG. 1: shows the electrophoretic mobility as a function of the pH of the gold nanoparticles, creatine, gold-creatine composite particles, and albumin.

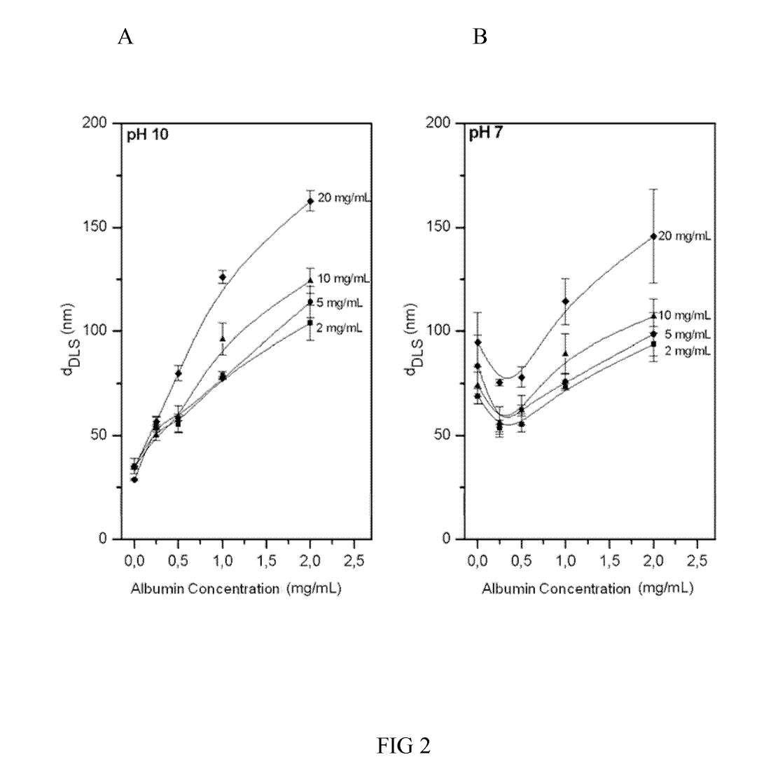

FIG. 2: shows the average hydrodynamic diameter of the composite particles as a function of the concentration of albumin, for different concentrations of creatine at pH 10 (FIG. 2A) and at pH 7.4 (FIG. 2B).

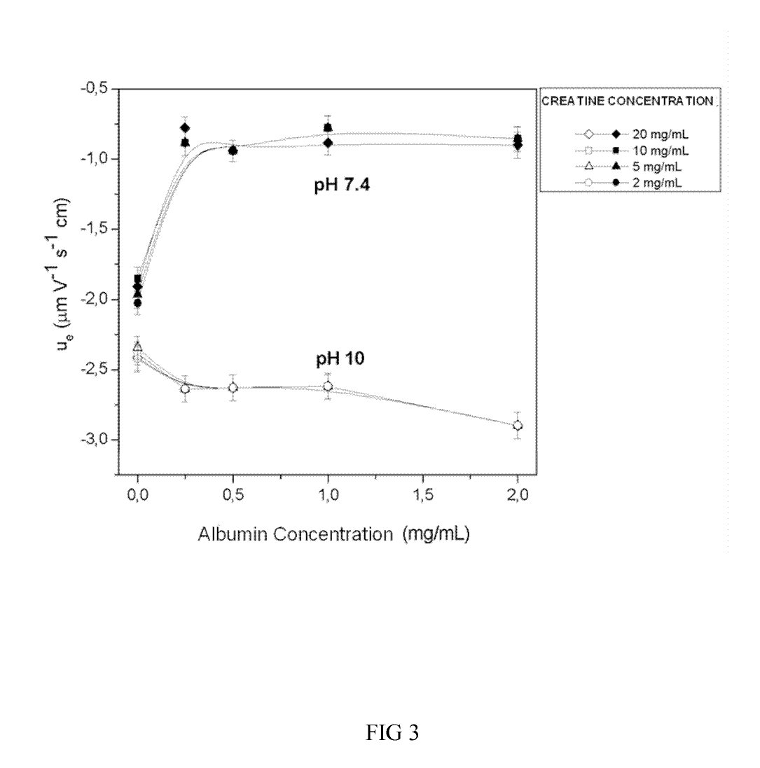

FIG. 3: shows the dependence of the electrophoretic mobility of the creatine-covered gold particles as a function of the concentration of albumin for different concentrations of creatine, at pH 10 and pH 7.4.

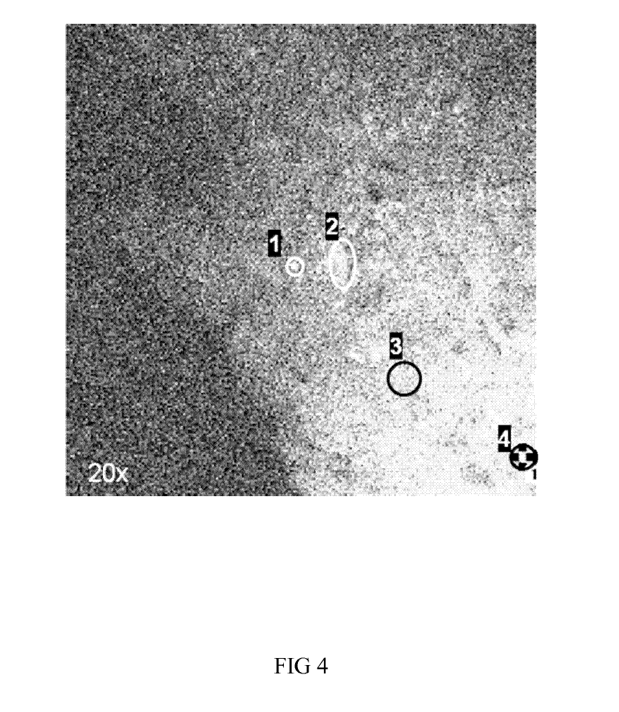

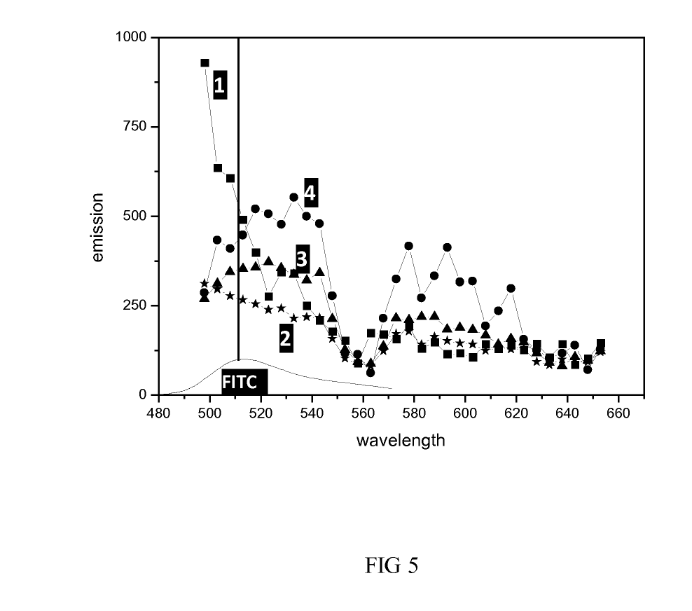

FIG. 4: 10 μm Brain slice of a mouse sacrificed 19 h after the nanogold coated with creatine/FITC-albumin was injected in the tail vein. The areas in the black and white circles are analyzed for their fluorescence spectra indicated in the diagram of FIG. 5 according to their number. The complete field of view is 636.5 μm×636.5 μm

FIG. 5: Diagram of the fluorescence emission spectra acquired from the image in FIG. 4. The numbers 1-4 are in accordance to the numbers of the region of interest (ROI) in FIG. 4. The spectrum indicated in the straight black line is the fluorescein isothiocyanate (FITC) emission. FITC is bound covalently to albumin which is absorbed onto the nanogold. The signal is from the sum of autofluorescence of the cells and FITC signal. In the spectra marked with squares and triangles the FITC signal is more evident than in those marked with stars or circles.

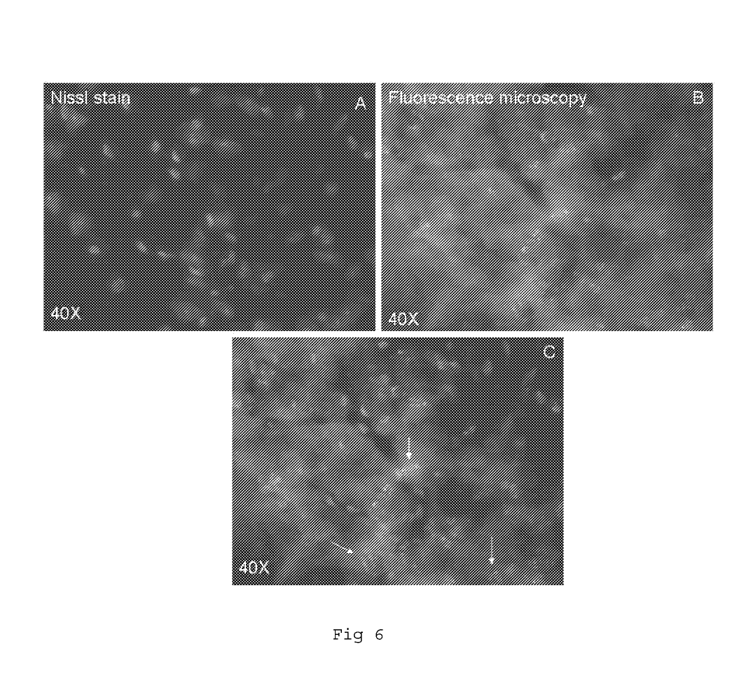

FIG. 6: Shows brain slices counterstained using Nissl staining for the visualization of the cell body (stains both neurons and glia). Cell body is seen by a counterstaining for the cells (FIG. 6A). Fluorescence signal (FIG. 6B) is a high spot-like intensity for the coated nanogold particles and a blurred fluorescence due to the autofluorescence of the cells. The merged image (FIG. 6C) shows that the particles are not only passing the blood brain barrier but also entering the cells (arrows).

### DESCRIPTION OF THE INVENTION

### Modes for Realizing the Invention

The present invention has been realized thanks to an accurate control of pH in the adsorption step of creatine on the surface of gold nanoparticles.

In a first preferred embodiment of the invention, the gold-creatine nanoparticle is covered by albumin.

Preferably, the gold core nanoparticle has a diameter ranging from 5 to 50 nm.

Once made, the nanoparticle according to the present invention has a hydrodynamic diameter comprised between 100 and 200 nm.

Another object of the present invention is a process for the preparation of the nanoparticle, comprising adding a dispersion of gold nanoparticles as a core to a creatine solution or creatine/albumin solution at a pH of both said dispersion and said solution higher than 9, preferably at least 11.

According to another object of the present invention, the nanoparticle is here described for use as a medicament.

In a preferred embodiment of the invention, the nanoparticle is provided for use as a medicament for treating stroke, in particular as neuroprotective molecules in stroke.

In another of its aspects, the present invention provides a gold-creatine nanoparticle, said particle is covered with a molecule capable of inducing the crossing of the blood-brain barrier.

In a preferred embodiment of the invention, said molecule is albumin.

In all its embodiments, the present invention provides said nanoparticle for use a medicament, in particular as a system of drug delivery.

Given the neuroprotective function of creatine, these pharmaceutical formulations may have an application in the treatment of ischemic stroke or other diseases leading to brain damage due to hypoxia as well as neurodegenerative diseases such as Alzheimer's disease, Parkinson's disease, and multiple sclerosis.

Therefore, it is another object of the present invention a pharmaceutical composition comprising an effective amount of nanoparticle as here described.

Pharmaceutical compositions are known and can be prepared with conventional processes, for example as described in Remington's Pharmaceutical Sciences, last edition, Mack Publishing and Co. Other examples of pharmaceutical compositions fit for the scopes of the invention can be found in US 2001/0006989,

The following example will further illustrate the invention.

### EXAMPLE

### Materials and Methods

### Materials

Deionized and filtered water (Milli-Q Academic, Millipore, France) was used in the preparation of all suspensions and solutions. The chemicals used were purchased from Sigma-Aldrich (Germany) with analytical quality, and were not further purified. Gold nanoparticles were synthesized according to the citrate thermal reduction method developed by Turkevich et al. [J. Turkevich, P. C. Stevenson, J. Hilier, Trans. Faraday Soc. 11 (1951) 55]. Briefly, a gold sol was prepared by adding 4.5 mg of sodium tetrachloroaurate (III) dihydrate in 25 mL of Milli-Q grade water, and 1 mL of a 1% sodium citrate tribasic dihydrate solution was rapidly added via a syringe into the boiling solution under vigorous stirring. The citrate ion acts both as reductant and stabilizer. It can clearly be seen after the addition of citrate how the color of the solution changes from yellowish to purple and finally wine red. After boiling for 20 minutes at 100° C., the solution was left to cool at room temperature under moderate magnetic stirring. The result is a stable dispersion of gold particles with an average hydrodynamic diameter of about 20 nm (standard deviation of 10%) and a real diameter of 15±1 nm. This value is in good agreement with the results reported by other authors elsewhere [J. Kunze, I. Burges, R. Nichols, C. Buess-Herman, J. Lipkowski, J. Elec. Chem., 599 (2007) 147-159].

Gold-creatine nanoparticles were synthesized by adding dropwise 1 mL of the previously obtained gold solution into a 0.5 mL solution of 2 mg/mL of creatine under moderate vortex rate. In order to have creatine molecules with a single charge, and for avoiding aggregation between the gold nanoparticles, the pH of both solutions was previously adjusted to 11.2. As result, the color of the solution remains red-pink, as it was originally. We noticed that, when the pH of both solutions is less than 11, the adsorption of creatine on the gold-nanoparticles surface is negligible, and the color of the solution turns to violet-blue, indicating the aggregation of gold nanoparticles. If the pH of the gold-creatine solution is decreased at this point a color change occurs indicating the aggregation of the creatine coated nanogold. Therefore, with the aim of improving the particle stability, increasing the amount of bound creatine, and inducing the transport through the blood brain barrier, an outermost layer of albumin is deposited in presence of the residual unbound creatine. The albumin-covered creatine-gold particles were obtained by adding 0.5 mL of the creatine-covered gold particles solution drop-wise into 1 mL of 1 mg/mL albumin solution at pH 10. This deposition is performed without a purification step to remove unbound creatine molecules. Then the pH was changed to physiological conditions (pH 7.4) because once the adsorption of albumin takes place, the nanoparticles are stable.

### Electrical Surface Characterization

Electrophoretic mobility (uₑ) measurements were performed in a Malvern Zetasizer 2000 apparatus (Malvern Instruments, England) at room temperature. Measurements were carried out 24 hours after the preparation of the suspensions, and the pH was readjusted immediately before the mobility was measured.

### Determination of the Concentration of Creatine Adsorbed

Adsorption of creatine from the nanogold solution was studied by HPLC method with an Agilent 1100 HPLC system (Agilent Technologies, Palo Alto, Calif.) equipped with a diode array detector, and a phase reverse column Atlantis T3 C18 (4.6×150 mm, 5 μm) from Waters (Waters Corp., Milford, USA). The samples were manually injected through a Rheodyne 7725i valve (Rheodyne, Cotati, USA) with a 50 μL loop.

The mobile phase was a phosphate buffer 10 mM, pH 5, with 5 mM 1-pentanesulfonic acid (Fluka) as ion pairing agent (coded as buffer A). All analysis of creatine were performed isocratically at a flow rate of 1 mL/min operating at a temperature of 30° C. The volume injected was 10 L. The eluate was simultaneously monitored for 10 minutes after the injection.

### Animal Model

Three C57 black female mice were treated intravenously (100 μl) as following:
Mouse n° 1: control (not treated).
Mouse n° 2: negative control (mouse treated EV with 5 nM of cyanine 5.5).
Mouse n° 3: experimental mouse (mouse treated with nanogold particles, coated with creatine and cyanine 5.5-labelled albumin, creatine: 50 mg bound to the particles).
   After the treatment mice were anesthetized using zolazepam plus xylazine (3.2 μl/gr intramuscular) and shaved in the belly and in the skull.

### NIR Time Domain Optical Imaging

Images from the three animal models were acquired at the NIR wavelength of 670 nm of excitation and 700 nm of emission. The laser power, integration time and scan step were optimized for each scan.

For all the animals images were acquired for both the abdomen (mouse upside up) and for the skull (mouse upside down) to distinguish the auto-fluorescence in the control animal with respect to the fluorescence emitted from the treated ones. The abdomen scans allow having information about the biodistribution of the probes in the whole body and their pharmacokinetics (liver metabolization and bladder excretion), while the skull scans allow assessing the capability of the tested compound to cross the blood-brain barrier.

Scans were acquired at time zero, 1 hour, 2 hours, 2 hours and 30 minutes, 3 hours, 3 hours and 30 minutes, 4 hours, 19, 24 and 48 hours.

Two days after the treatments, mice were sacrificed by cervical dislocation, the brains were explanted, washed in PBS and tissue fluorescence was analyzed ex vivo.

### Fluorescence Microscopy

The crossing of the blood brain barrier of the nanoparticles was shown in two different ways. Brains were sliced in saggital orientation with a in 10 μm sections and the tissue slices were thaw-mounted onto surface-treated glass slides. For the first technique, the slices were analyzed with confocal microscope and the distribution of particles was localized and identified by spectral analysis of the fluorescence emission. The images were acquired with a 20× objective. After the localization of the brain regions with accumulation of the coated nanogold the cells in that region were imaged in more detail. For this, the brain slices were counterstained using Nissl staining for the visualization of the cell body (stains both neurons and glia). Images were acquired in an epifluorescence microscope at 488 nm excitation wavelength and in a wide field white light microscope, both with an 40×10.75 objective.

### Results:

### Effect of the pH

In FIG. 1. electrophoretic mobility as a function of the pH of the gold nanoparticles, creatine, gold-creatine composite particles, and albumin are shown. As observed, the gold nanoparticles present a negative electrophoretic mobility (uₑ) over the whole range of pH, characteristic of the citrate molecules adsorbed on them, becoming more negative with pH increase. Creatine electrophoretic mobility is also negative in the whole range of pH, although uₑ is very close to zero under acidic conditions. On the contrary, albumin molecules present an isoelectric point between pH 4.5-5, becoming more negative with pH increase.

This electrokinetic technique is a very useful tool for qualitatively checking the coating efficiency. The electrophoretic mobility of the creatine-covered gold composite particles is negative for the whole range of pH studied, it decreases as the pH becomes more basic and, as it can be seen, from an electrokinetic point of view, the results are qualitatively similar to the values obtained for the creatine molecules specially at basic pHs.

The adsorption of creatine by the gold particles, as well as albumin as outermost layer has also been followed by dynamic light scattering measurements. FIG. 2 shows the average hydrodynamic diameter of the composite particles as a function of the concentration of albumin, for different concentrations of creatine at pH 10 (FIG. 2A) and at pH 7.4 (FIG. 2B). It is worthy to mention that, at pH 10, the measured hydrodynamic diameter for the gold nanoparticles is (21±3) nm, and a good indication of the adsorption of creatine on their surface is the increase of the diameter size to (31±4) nm. The instability mentioned above of the creatine-covered gold composite particles can be clearly appreciated by comparing the size of the particles at pH 10 and at pH 7. As it can be seen, when the pH is decreased to 7.4, the size of the particles increases, the larger it is, the higher is the concentration of creatine (i.e. for concentrations of creatine of 20 mg/mL, the hydrodynamic diameter at pH 10: d_{pH10}≈30 nm, increases to d_{pH7.4}≈90 nm, when pH is 7.4). This increase in size comes together with a change in the color of the solution, as it was mentioned before. The adsorption of albumin can also be tracked by comparing the size of the particles: there is a significant enlargement on the size of the particles, as the concentration of albumin is increased.

As in the case of the gold-creatine, uₑ measurements were performed on the albumin-covered creatine-gold composite particles. FIG. 3 shows the dependence of the electrophoretic mobility of the creatine-covered gold particles as a function of the concentration of albumin for different concentrations of creatine, for pH 10 and pH 7.4. As it can be seen: (i) in the absence of albumin, the uₑ of the creatine-gold particles do not present significant differences for the concentrations of creatine studied; as in FIG. 1, the more basic is the pH, the more negative is the uₑ; (ii) absorption of albumin by creatine-covered particles is clearly shown by a significant difference in the electrophoretic mobility; as it can be seen, at pH 10 there is a slight decrease of uₑ compared to the ones obtained in the absence of the albumin; on the contrary, at pH 7.4, the lower charge of the albumin showed in FIG. 1, contributes to screen the surface charge of the creatine-covered particles, compared to the one obtained without albumin; (iii) in any case, for both pH conditions, the effect of the concentration of albumin is also very little, as we have observed for the concentration of creatine. These results point out the clear protecting effect exerted by the protein when it covers the creatine-covered gold particles.

### In Vivo Distribution

It is noted that fluorescence lifetime increases as a function of time in the brain region, indicating the presence of the particles inside the brain.

The analysis with microscope shows that the particles according to the present invention are able to cross the blood-brain membrane.

The detection of nanoparticles in the brain tissue after crossing the blood brain barrier

The presence of the particles in the brain were detected with different techniques in order to be sure that they are really passing the blood brain barrier. First hint of particle accumulation in the brain region are given by the NIR (near infrared) fluorescence distribution in vivo of a dye covalently bound to albumin coating on the nanogold surface. In this context it is noteworthy that albumin alone is not able to cross the blood brain barrier. In the next step the brains of the injected mice were removed after sacrificing the animals 19 h or longer after injection. In the sliced brains the particles and their overall distribution was imaged by fluorescence microscopy visualizing again the particle coating but on a cellular (FIG. 4) and even sub-cellular level. The problem in the visualization of the fluorophore is the broad autofluorescence spectrum of the brain neurons. In order to be sure that the signal is really related to the fluorophore and not exclusively autofluorescence of the compound of cell metabolism the emission spectrum is recorded for the region of interest (ROI). Four ROIs are marked with a ring in FIG. 4 and in the diagram in FIG. 5 the resulting emission spectra are depicted. For reasons of better understanding the emission spectra of pure FITC (fluorescein isothiocyanate) was added. The maximum emission for the FITC is at 515 nm. The ROIs 1, 3, and 4 show a significant amount of emission in that range overlaid with the emission of the autofluorescence of the cells.

By means of a confocal microscope with spectral detection unit the brain slices were imaged with high resolutions and regions with high fluorescence signal (FIG. 4) were analyzed for the fluorescence emission spectrum (FIG. 5) in order to identify the areas of the brain with a high emission for FITC (fluorescein isothiocyanate) which indicates the presence of albumin used as coating for the nanogold. After the localization of the brain regions with accumulation of the coated nanogold the cells in that region were imaged in more detail. For this, the brain slices were counterstained using Nissl staining for the visualization of the cell body (stains both neurons and glia). In this way the cell body clearly can be seen by a counterstaining for the cells (FIG. 6A) while the fluorescence signal (FIG. 6B) gives a high spot-like intensity for the coated nanogold particles and a blurred fluorescence due to the autofluorescence of the cells. In the merged image (FIG. 6C) it can be seen clearly that the particles are not only passing the blood brain barrier but also entering the cells (arrows). This is important for the release of the neuroprotective molecule, creatine directly to the site where it is needed.

### POTENTIAL APPLICATION

### Industrial Application

The present invention relates to the medical field, in particular to pharmaceutical formulations for the selective release of drugs that can cross the blood-brain barrier and reach the sites affected by brain stroke. Given the neuroprotective function of creatine, these pharmaceutical formulations may have an application in the treatment of ischemic stroke or other diseases leading to brain damage due to hypoxia as well as neurodegenerative diseases such as Alzheimer's disease, Parkinson's disease, and multiple sclerosis.

1. Gold-creatine nanoparticle.

2. A nanoparticle according to claim 1 which is albumin-covered.

3. A nanoparticle according to claim 1, wherein the gold core nanoparticle has a diameter ranging from 5 to 50 nm.

4. A nanoparticle according to claim 1, wherein the diameter of said nanoparticle ranges from 100 to 200 nm.

5. A process for the preparation of the nanoparticle of claim 1 comprising adding a dispersion of gold nanoparticles as core to a creatine solution at a pH of both said dispersion and said solution higher than 9.

6. A process according to claim 5, wherein said gold core nanoparticles have a diameter ranging from 5 to 50 nm.

7. Nanoparticle of claim 1 for use as medicament.

8. Nanoparticle of claim 7 for use as medicament for the treatment of stroke.

9. Nanoparticle of claim 7 for use as neuroprotective in stroke.

10. Nanoparticle of claim 7 for use as medicament in diseases leading to brain damage due to hypoxia as well as neurodegenerative diseases such as Alzheimer's disease, Parkinson's disease, and multiple sclerosis.

11. Creatine-gold nanoparticle covered with a molecule capable of crossing the blood-brain barrier.

12. Nanoparticle according to claim 11, wherein said molecule is albumin.

13. Nanoparticle of claim 11 for use as medicament.

14. Nanoparticle of claim 11 for use as drug-delivery system.

15. Pharmaceutical composition comprising an effective amount of nanoparticle of claim 1.
